(19) United States
(12) Patent Application Publication
(10) Pub. No.: US 2016/0263169 A1
(43) Pub. Date: Sep. 15, 2016

(54) COMPOSITION COMPRISING AN EXTRACT OF COMBINED HERB CONSISTING OF HEAT PROCESSED GINSENG, HOUTTUYNIAE HERBA, PERILLA LEAF AND TEA LEAF OR THE PROCESSED EXTRACT THEREOF AS AN ACTIVE INGREDIENT SHOWING HAIR-STIMULATING ACTIVITY AND PREVENTING ACTIVITY FROM HAIR LOSS, AND THE USE THEREOF.

(71) Applicant: GINSENG SCIENCE INC., Seoul (KR)

(72) Inventors: Bok Deuk KIM, Seoul (KR); Sung Hyang KIM, Seoul (KR); Yang Beom LEE, Seoul (KR); Man Ki PARK, Gyeonggi-do (KR); Yong Jae LEE, Gyeonggi-do (KR)

(21) Appl. No.: 14/830,674

(22) Filed: Aug. 19, 2015

(30) Foreign Application Priority Data
Mar. 11, 2015 (KR) 10-2015-33530

(51) Int. Cl. A61K 36/258; A61K 36/535; A61K 36/82; A61K 36/78

(52) U.S. Cl. CPC A61K 36/258; A61K 36/78; A61K 36/535; A61K 36/82

(57) ABSTRACT

The present invention relates to a composition comprising the extract of combined herbs consisting of heat processed ginseng, houttuyniae herba, perilla leaf and tea leaf or the processed extract thereof as active ingredients for preventing and treating hair baldness and stimulating activity of hair growth. The inventive combined extract showed more potent hair-growth promoting activity and synergistic effect than the precedent invention(s), for example, a fermented extract of Houttuyniae Herba, Perilla leaf and Tea leaf disclosed in Korea Patent Publication No, 10-2014-0114492 (A1), through various animal model experiments such as the growth rate test using by C57BL/6 mouse, the proliferating effect on the growth of HFDPC etc and additionally, more favorable advantage than the precedent invention(s), for example, the easiness in controlling the prescribed dosage by dint of the final form of the inventive extract, i.e., concentrated solid form comparing with the solution type of the final form disclosed in precedent invention.

### TECHNICAL FIELD

The present invention relates to a composition comprising the extract of combined herbs consisting of heat processed ginseng, Houttuyniae Herba, Perilla leaf and Tea leaf or the processed extract thereof as an active ingredient showing hair-growth stimulating activity and preventing activity from hair loss, and the use thereof.

### BACKGROUND ART

Hair follicle, a very complex organ, consists of an inner root sheath (IRS), outer root sheath (ORS), hair shaft, hair matrix cell (Paus et al., J. Invest. Dermatol., 113, pp 523-532, 1999), and dermal papilla cell, which plays an important role in hair growth among various kinds of the cells forming the hair follicle (Ferraris et al., Exp. Cell Res., 10, pp 37-46, 1997).

Hair follicle develops through the interaction between the mesenchymal cell and epithelial cell during an embryogenesis. Normal hair growth consists of repeated three phases, i.e., anagen, catagen and telogen stages (Paus et al., N. Engl. J. Med., 341, pp 491-497, 1999). Especially, human hair has the longer growth period than any other animal hair or the other part hairs of the human body (anagen, 2-5 years; catagen, several days-several weeks; telogen, three months). During the anagen stage, the cell consisting of hair follicle including the keratinocytic cell of matrix surrounding the dermal papilla, proliferates and grows hair. During the catagen stage, within the hair follicle, apoptosis, condensation of the dermal papilla, and the reconstitution of extracellular matrix serially occur (Paus et al., J. Invest. Dermatol., 113, pp 523-532, 1999) to stop the cell division and slow the hair growth. The hair follicle at telogen stage falls out except the permanently lasting parts comprising the bulge region of hair follicle at the state of quiescence of growth to restart new anagen (Hardy M H., Trends Genet., 8, pp 55-61, 1992).

Several drugs promoting hair growth approved by the Food and Drug Administration (FDA) have been developed, for example, ‘Minoxidil’ (Buhl et al., J. Invest. Dermatol., 92(3), pp 315-320, 1989) and ‘Finasteride’ (Van Neste et al., Br. J. Dermatol., 143(4), pp 804-810, 2000).

However, those drugs have been reported to show several disadvantages, for example, limitation to use such as effective only in the alopecia of specific area; inconvenience in use such as long-term administration (about 6 month to 1 year), subsequently topical administration, for example, twice a day, etc; various adverse responses in case of long-term use such as a decrease of sexual desire, erectile dysfunction, increased risk to give birth to congenitally anomalic babies, pruritus, irritation etc (J. Korean Med. Assoc., Vol., 55(5), pp 475-483, 2012; J. Ginseng Research, 33(3), pp 223, 2009).

As an alternative approach, there have been attempts to develop new therapy using herb extract having hair growth stimulating effect: for example, an extract of fermented herbs of Houttuyniae Herba, Perilla leaf and Tea leaf disclosed in Korea Patent Publication No, 10-2014-0114492 (A1) etc.

However, there have been still needed to develop new drugs or substances showing more potent activity without adverse action till now.

There are many species of Panax genus plants belonged to Araliaceae, for example, Panax ginseng distributed or cultivated in far-eastern Asia region, Panax quinquefolius in America and Canada, Panax Notoginseng in China, Panax trifolia in eastern region of north America, Panax vietnamensis in Vietnam, Panax elegatior, Panax wangianus and Panax bipinratifidus etc.

Recently, there have been several attempts to strengthen pharmacological effects among ginseng by modifying the method of ginseng processing, for example, Park et al developed new methods for preparing a processed ginseng under specific high temperature and high pressure as disclosed in Korean Patent Registration No. 192678 and U.S. Pat. No. 5,776,460, which changes main ginseng saponins such as ginsenosides Rb1, Rb2, Rc and Rd, into new saponins such as ginsenosides Rg3, Rg5, Rk1, Rk2, Rk3, Rs1, Rs2, Rs3, F4, Rh2, Rh4 and compound K showing new and more potent pharmacological effects, for examples, anti-oxidative activity, anti-cancer activity and alleviating activity of blood circulation etc (Kim W Y et al., J. Nat. Prod., 63(12), pp 1702-1704; Kwon S H et al., J. Chromatogr. A., 921(2), pp 335-339, 2001). Especially, ginsenosides Rg3, Rg5 and Rk1 has been known to show most potent pharmacological activities among them, for example, neuro-protective activity, anti-dementia activity, memory-enhancing activity etc (Yang L L et al., J. Pharm. Pharmacol., 61, pp 375-380, 2009; Bao H. Y., et al., Arch. Pharm. Res., 28(3), pp 335-342, 2005). Accordingly, these new ginsenosides can be produced in the root, stem or leaf of any Panax genus plants such as Panax ginseng, Panax quinquefolia, Panax notoginseng, Panax japonica, Panax trifolia, Panax pseudoginseng, Panax vietnamensis, Panax elegatior, Panax wangianus and Panax bipinratifidus which contains dammarane glycoside through the processing method of Park et al (Korean Patent Registration No. 192678 and U.S. Pat. No. 5,776,460).

Houttuyniae Herba, whole body of Houttuynia cordata THUNB belonged to Saururaceae, is distributed to Korea. It has been reported to comprise various ingredients, for example, decanoyl acetaldehyde, pinene, linalool, camphene, limonene, cordarine, quercitrin, isoquercitrin etc and to show various medicinal effect, for example, anti-bacterial activity, anti-viral activity and diuretic activity etc (B. S. CHUNG et al. Dohaehyangyakdaesajeon, Youngrim Press, pp. 812-813, 1998).

Perilla leaf, a leaf of Perilla frutescens BRITT. var acuta KUDO or Perilla frutescens BRITT. var crispa DECNE belonged to Labiatae is distributed in Korea. It has been reported to comprise various ingredients, for example, perilaldehyde, limonene, arginine, cumic acid, isogomaketone, perilla alcohol etc and to treat common cold (B. S. CHUNG et al. Dohaehyangyakdaesajeon, Youngrim Press, pp. 855-857, 1998).

Tea leaves, a leaf of Camellia sinensis O. KTZE belonged to Theaceae is distributed in Asian countries including Korea. It has been reported to comprise various ingredients, for example, caffeine, theobromine, theophylline, xanthine, tannin etc and to show CNS stimulating activity, vasodilating activity, diuretic activity etc (B. S. CHUNG et al. Dohaehyangyakdaesajeon, Youngrim Press, pp. 403-405, 1998).

However, there has been not reported or disclosed about the hair growing activity of the extract of combined herbs consisting of heat processed ginseng, Houttuyniae Herba, Perilla leaf and Tea leaf or the processed extract thereof in any of above cited literatures, the disclosures of which are incorporated herein by reference.

Therefore, the inventors of the present invention have developed to find more potent treating agent for alopecia than the precedent invention(s), for example, a fermented extract of Houttuyniae Herba, Perilla leaf and Tea leaf disclosed in Korea Patent Publication No, 10-2014-0114492 (A1) till now.

Through the investigation, the present inventors have found novel inventive combination of the present invention showed unexpectedly more potent treating effect on alopecia than the precedent invention(s), which is evaluated by various animal model experiments such as the growth rate test using by C57BL/6 mouse, the proliferating effect on the growth of HFDPC etc and finally completed the present invention by confirming the potent activity of hair growth.

These and other objects of the present invention will become apparent from the detailed disclosure of the present invention provided hereinafter.

### DISCLOSURE

### Technical Problem

The present invention relates to a pharmaceutical composition comprising the extract of combined herbs consisting of heat processed ginseng, houttuyniae herba, perilla leaf and tea leaf or the processed extract thereof as active ingredients for preventing or treating hair baldness and stimulating activity of hair growth and the use thereof.

The present invention also relates to a use of the extract of combined herbs consisting of heat processed ginseng, houttuyniae herba, perilla leaf and tea leaf or the processed extract thereof for manufacture of medicament employed for preventing and treating hair baldness and stimulating activity of hair growth in human or mammal.

The present invention also relates to a method for treating hair baldness and stimulating activity of hair growth in a mammal or animal suffering from said diseases comprising administering an effective amount of the extract of combined herbs consisting of heat processed ginseng, houttuyniae herba, perilla leaf and tea leaf or the processed extract thereof, together with a pharmaceutically acceptable carrier to said mammal or animal in need thereof.

The present invention also relates to a health functional food comprising the extract of combined herbs consisting of heat processed ginseng, houttuyniae herba, perilla leaf and tea leaf or the processed extract thereof as active ingredients for alleviating or preventing hair baldness and stimulating activity of hair growth and the use thereof.

The present invention also relates to a cosmetic composition comprising the extract of combined herbs consisting of heat processed ginseng, houttuyniae herba, perilla leaf and tea leaf or the processed extract thereof as active ingredients for preventing and improving hair baldness and stimulating activity of hair growth and the use thereof.

### Technical Solution

It is an object of the present invention to provide a pharmaceutical composition comprising the extract of combined herbs consisting of heat processed ginseng, houttuyniae herba, perilla leaf and tea leaf or the processed extract thereof as active ingredients for preventing and treating hair baldness and stimulating activity of hair growth.

The term “heat processed ginseng” disclosed herein comprises a root, leaf, fruit, or rhizome of a processed ginseng such as red ginseng or, a processed ginseng produced by heating at the temperature ranging from 80 to 200° C., preferably, 100 to 150° C. for the period ranging from 0.5 to 78 hours, preferably, 1 to 24 hours so as to make a ratio of ginsenoside (Rg3+Rg5+Rk1) to (Rb1+Rb2+Rc+Rd) of over 1.0; or a wild ginseng such as white ginseng, of which ginseng is selected from Panax ginseng, Panax quinquefolia, Panax notoginseng, Panax japonica, Panax trifolia, Panax pseudoginseng, Panax vietnamensis, Panax elegatior, Panax wangianus or Panax bipinratifidus, preferably, Panax ginseng

The term “extract” disclosed herein includes polar solvent soluble extract, for example, the extract soluble in at least one solvent selected from consisting of water, ethanol, butanol, propanol, acetone, ethylacetate, hexane, butyleneglycol, propyleneglycol, hydrobutyleneglycol, hydropropyleneglycol, hydroglycerin, or the mixture thereof, preferably, water, methanol, ethanol or the mixture thereof, more preferably, the mixture solvent with water and ethanol, more and more preferably from 60 to 90% ethanol.

The term “extract of combined herbs” disclosed herein comprises the extract of combined herbs consisting of heat processed ginseng, houttuyniae herba, perilla leaf and tea leaf with mixed ratio of 1-10:1-10:1-10:1-10 (w/w), preferably, 1-5:1-5:1-5:1-5 (w/w), more preferably, 1-3:1-3:1-3:1-3 (w/w).

The term “processed extract” disclosed herein includes “a processed extract with resin, preferably, a processed extract with reverse phase partition chromatography or any chromatography using by any resin as a stationary phase which can retain non-polar substance while eluting polar substance, for example, Sephadex resin such as Sephadex, Sephadex LH20, Sephadex G-25, Sephadex G-10, Sepharose, Superdex, methylacrylate resin, carboxymethyl cellulose, sulphopropyl cellulose, carboxymethyl Sephadex, sulphopropyl Sephadex, carboxymethyl Sepharose, sulphopropyl Sepharose and the like; reverse polymer resin using by Stylene-divinylbenzen co-polymer such as Polymer X, HP20, PRP-h1 Polymer and the like or Methacrylate support resin etc and using at least one solvent selected from water, acetonitrile, lower alcohol such as methanol, ethanol, butanol etc, tetrahydrofuran (THF) or the mixture thereof, preferably, water, lower alcohol such as methanol, ethanol, butanol etc, or the mixture thereof, more preferably, water or the mixture solvent of water and ethanol, more and more preferably, water or the mixture solvent of water and methanol with starting from 90:10(v/v) to 60:40(v/v) to elute polar substance and retain non-polar substance as a mobile phase.

The term “baldness disorder” disclosed herein comprises androgenic alopecia, alopecia senile, alopecia areata and the like.

Hereinafter, the present invention is described in detail.

The herbs, which can be used in the present invention, include the same genus plants which would be apparent to those skilled in the art and have been used for identical or similar purpose and can be substituted for the prevention and treatment of the diseases.

The inventive combined extract of the present invention can be prepared by the followings;

For example, the inventive extract of the present invention can be prepared by the method comprising the step of; heating a ginseng with high temperature ranging from 80 to 200° C., preferably, 100 to 150° C. for the period ranging from 0.5 to 78 hours, preferably, 1 to 24 hours, so as to make a ratio of ginsenoside (Rg3+Rg5+Rk1) to (Rb1+Rb2+Rc+Rd) of over 1.0 to afford a heat processed ginseng at the 1^{st} step; mixing the a heat processed ginseng with Houttuyniae herba, Perilla leaf and Tea leaf with mixed ratio of 1-10:1-10:1-10:1-10 (w/w), preferably, 1-5:1-5:1-5:1-5 (w/w), more preferably, 1-3:1-3:1-3:1-3 (w/w) to afford combined herbs at the 2^{nd} step; mixing the combined herbs with at least one solvent selected from consisting of water, ethanol, butanol, propanol, acetone, ethylacetate, hexane, butyleneglycol, propyleneglycol, hydrobutyleneglycol, hydropropyleneglycol, hydroglycerin, or the mixture thereof, preferably, water, methanol, ethanol or the mixture thereof, more preferably, the mixture solvent with water and ethanol, more and more preferably from 60 to 90% ethanol and subjecting to extraction selected from reflux extraction, cold water extraction, ultra-sonication or other conventional extraction, preferably reflux extraction with the temperature ranging from 0 to 150° C., preferably, 50 to 120° C. for the period ranging from 0.1 to 48 hours, preferably, 0.5 to 12 hours repeatedly, preferably, 1-10 times, more preferably, 2-7 times to afford the extract of combined herbs at the 3^{rd} step; filtering and concentrating the extract at the 4^{th} step; suspending the concentrating extract into water to afford the suspend solution at the 5^{th} step; performing the suspended solution with a reverse phase partition chromatography or any chromatography using by any resin as a stationary phase which can retain non-polar substance while eluting polar substance, for example, Sephadex resin such as Sephadex, Sephadex LH20, Sephadex G-25, Sephadex G-10, Sepharose, Superdex, methylacrylate resin, carboxymethyl cellulose, sulphopropyl cellulose, carboxymethyl Sephadex, sulphopropyl Sephadex, carboxymethyl Sepharose, sulphopropyl Sepharose and the like; reverse polymer resin using by Stylene-divinylbenzen co-polymer such as Polymer X, HP20, PRP-h1 Polymer and the like or Methacrylate support resin etc and using at least one solvent selected from water, acetonitrile, lower alcohol such as methanol, ethanol, butanol etc, tetrahydrofuran (THF) or the mixture thereof, preferably, water, lower alcohol such as methanol, ethanol, butanol etc, or the mixture thereof, more preferably, water or the mixture solvent of water and ethanol, more and more preferably, water or the mixture solvent of water and methanol with starting from 90:10(v/v) to 60:40(v/v) as a mobile phase to elute polar substance and to collect non-polar soluble fraction from the extract at the 6^{th} step; and concentrating non-polar soluble fraction to afford the processed extract of combined herbs consisting of heat processed ginseng, houttuyniae herba, perilla leaf and tea leaf.

Accordingly, the present invention also provides a method for preparing an extract of combined herbs consisting of heat processed ginseng, houttuyniae herba, perilla leaf and tea leaf or the processed extract thereof, comprising the steps of; heating a dried ginseng with high temperature ranging from 80 to 200° C., preferably, 100 to 150° C. for the period ranging from 0.5 to 78 hours, preferably, 1 to 24 hours, so as to make a ratio of ginsenoside (Rg3+Rg5+Rk1) to (Rb1+Rb2+Rc+Rd) of over 1.0 to afford a heat processed ginseng the 1^{st} step; mixing the a heat processed ginseng with houttuyniae herba, perilla leaf and tea leaf with mixed ratio of 1-10:1-10:1-10:1-10 (w/w), preferably, 1-5:1-5:1-5:1-5 (w/w), more preferably, 1-3:1-3:1-3:1-3 (w/w) to afford combined herbs at the 2^{nd} step; mixing the combined herbs with at least one solvent selected from consisting of water, ethanol, butanol, propanol, acetone, ethylacetate, hexane, butyleneglycol, propyleneglycol, hydrobutyleneglycol, hydropropyleneglycol, hydroglycerin, or the mixture thereof, preferably, water, methanol, ethanol or the mixture thereof, more preferably, the mixture solvent with water and ethanol, more and more preferably from 60 to 90% ethanol and subjecting to extraction selected from reflux extraction, cold water extraction, ultra-sonication or other conventional extraction, preferably reflux extraction with the temperature ranging from 0 to 150° C., preferably, 50 to 120° C. for the period ranging from 0.1 to 48 hours, preferably, 0.5 to 12 hours repeatedly, preferably, 1-10 times, more preferably, 2-7 times to afford the extract of combined herbs at the 3^{rd} step; filtering and concentrating the extract at the 4^{th} step; suspending the concentrating extract into water to afford the suspend solution at the 5^{th} step; performing the suspended solution with a reverse phase partition chromatography or any chromatography using by any resin as a stationary phase which can retain non-polar substance while eluting polar substance, for example, Sephadex resin such as Sephadex, Sephadex LH20, Sephadex G-25, Sephadex G-10, Sepharose, Superdex, methylacrylate resin, carboxymethyl cellulose, sulphopropyl cellulose, carboxymethyl Sephadex, sulphopropyl Sephadex, carboxymethyl Sepharose, sulphopropyl Sepharose and the like; reverse polymer resin using by Stylene-divinylbenzen co-polymer such as Polymer X, HP20, PRP-h1 Polymer and the like or Methacrylate support resin etc and using at least one solvent selected from water, acetonitrile, lower alcohol such as methanol, ethanol, butanol etc, tetrahydrofuran (THF) or the mixture thereof, preferably, water, lower alcohol such as methanol, ethanol, butanol etc, or the mixture thereof, more preferably, water or the mixture solvent of water and ethanol, more and more preferably, water or the mixture solvent of water and methanol with starting from 90:10(v/v) to 60:40(v/v) as a mobile phase to elute polar substance and to collect non-polar soluble fraction from the extract at the 6^{th} step; and concentrating non-polar soluble fraction to afford the processed extract of combined herbs consisting of heat processed ginseng, houttuyniae herba, perilla leaf and tea leaf of the present invention.

It has been confirmed that the inventive combined extract showed more potent hair-growth promoting activity and synergistic effect than the precedent invention(s), for example, a fermented extract of Houttuyniae Herba, Perilla leaf and Tea leaf disclosed in Korea Patent Publication No, 10-2014-0114492 (A1), through various animal model experiments such as the growth rate test using by C57BL/6 mouse, the proliferating effect on the growth of HFDPC etc and additionally, found more favorable advantage than the precedent invention(s), for example, the easiness in controlling the prescribed dosage by dint of the final form of the inventive extract, i.e., concentrated solid form comparing with the solution type of the final form disclosed in precedent invention.

Inventive composition of the present invention has no toxicity and adverse effect therefore can be used with safe.

The present invention also provided a use of the extract of combined herbs consisting of heat processed ginseng, houttuyniae herba, perilla leaf and tea leaf or the processed extract thereof for the preparation of therapeutic agent for the treatment and prevention of baldness disorder in mammal or human.

The present invention also provided a pharmaceutical composition comprising the extract of combined herbs consisting of heat processed ginseng, houttuyniae herba, perilla leaf and tea leaf or the processed extract thereof, and a pharmaceutically acceptable carrier thereof as an active ingredient for treating and preventing baldness disorder.

It is an object of the present invention to provide a method of treating or preventing baldness disorder in mammal or human in need thereof comprising administering to said mammal or human with an effective amount of the extract of combined herbs consisting of heat processed ginseng, houttuyniae herba, perilla leaf and tea leaf or the processed extract thereof, together with a pharmaceutically acceptable carrier thereof.

The inventive composition for preventing baldness disorder and stimulating hair growth may comprise the above-described extract as 0.1˜50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co., Easton Pa.).

Hereinafter, the following formulation methods and excipients are merely exemplary and in no way limit the invention.

Therefore, the present invention may be prepared by mixing the inventive extract with the pharmaceutically acceptable carriers, adjuvants or diluents as pharmaceutical composition for prevention and treatment of purposed disease or disorders.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinyl pyrrlidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like.

The desirable dose of the inventive extract varies depending on the condition and the weight of the subject, severity, drug form, route and period of administration, and may be chosen by those skilled in the art. However, in order to obtain desirable effects, it is generally recommended to administer at the amount ranging from 0.0001 to 100 mg/kg, preferably 0.001 to 100 mg/kg by weight/day of the inventive compounds of the present invention. The dose may be administered in single or divided into several times per day.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intrathecal, epidural or intracerebroventricular injection.

The present invention provides a health functional food comprising the extract of combined herbs consisting of heat processed ginseng, houttuyniae herba, perilla leaf and tea leaf or the processed extract thereof, as an active ingredient for preventing or alleviating hair baldness and stimulating activity of hair growth.

In a preferred embodiment, it is the other object of the present invention to provide a health functional food or health care food comprising the extract of combined herbs consisting of heat processed ginseng, houttuyniae herba, perilla leaf and tea leaf or the processed extract thereof, for preventing or alleviating hair baldness and stimulating activity of hair growth, together with a sitologically acceptable additive.

The term “a sitologically acceptable additive” disclosed herewith comprises the additive which can be conventionally available well-known in the art, for example food additive lists published on U.S. Food and Drug Administration (See, www.fda.gov/food).

The health functional food composition for preventing and improving purposed diseases could contain about 0.01 to 95 w/w %, preferably 0.5 to 80 w/w % of the above crude extract based on the total weight of the composition.

Above described the crude drug composition therein can be added to food, additive or beverage for prevention and improvement of purposed diseases. For the purpose of preventing and improving purposed diseases, wherein, the amount of above described crude drug composition in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described extract as an essential component in the indicated ratio, there is no particular limitation on the other liquid component wherein the other component can be various deodorant or natural carbohydrate etc such as conventional beverage. Examples of aforementioned natural carbohydrate are monosaccharide such as glucose, fructose etc; disaccharide such as maltose, sucrose et al.; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartame etc, may be useful favorably. The amount of above described natural carbohydrate generally ranges from about 1 to 20 g, preferably 5 to 12 g in the ratio of 100 ml of present beverage composition.

The other components than aforementioned composition are various nutrients, a vitamin, a mineral or and electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition

It is an object of the present invention to provide a cosmetic composition comprising an extract of combined herbs consisting of heat processed ginseng, Houttuyniae Herba, Perilla leaf and Tea leaf or the processed extract thereof as active ingredients for preventing or improving hair baldness and stimulating activity of hair growth.

The other components may be a mixture of the ingredients of a conventional cosmetic composition well known in the art.

Cosmetic formulations containing above composition may be prepared in any form such as hair tonic, hair conditioner, hair essence, hair lotion, hair nutrient lotion, hair shampoo, hair rinse, hair treatment, hair cream, hair nutrient cream, hair was, hair moisture cream, hair massage cream, hair aerosol, hair pack, hair nutrient pack, hair soap, hair cleaning foam, pomade, hair drying agent, hair care agent, hair dyeing agent, hair permanent wave agent, hair bleaching agent, hair gel, hair glaze, hair dressing pomade, hair lacquer, hair moisturizer, hair mousse, eyebrows nutrient, eyelash nutrient, hair spray, astringent, nutrient lotion, nutrient cream, massage cream, essence, pack, foundation, cleansing water, soap, treatment, beauty solution and the like.

The cosmetic composition of the present invention can comprises additional additives selected from the group consisting of water soluble vitamin, lipid soluble vitamin, peptide polymer, polysaccharide polymer, sphingolipid and sea-weed extract.

Preferable water soluble vitamins are any one which can be mixed with cosmetic, however, various vitamin such as vitamin B₁, B₂, B₆, pyridoxine, pyridoxine HCl, vitamin B₁₂, pantothenic acid, nicotinic acid, nicotinamide, folic acid, vitamin C, vitamin H etc, the salt thereof such as thiamin HCl salt, ascorbic acid Na salt etc or their derivatives such as ascorbic acid-2-phosphonic acid Na salt, ascorbic acid-2-phosphonic acid Mg salt are preferable and those can be obtained by conventional method such as microbial conversion method, purification method from the microbial cultivates, enzymatic method or chemical synthetic method.

Preferable lipid soluble vitamins are any one which can be mixed with cosmetic, however, various vitamin such as vitamin A, D₂, D₃, E dl-tocopherol, d-tocopherol, l-tocopherol and their derivatives such as palmitic acid ascorbate, stearic acid ascorbate, dipalmitic acid ascorbate, acetic acid-dl-tocopherol, nicotinic acid dl-tocopherol vitamin E, dl-pantothenyl alcohol, D-pantothenyl alcohol, pantothenyl ethylether etc. including the lipid soluble vitamin used in examples of present invention are preferable and those can be obtained by conventional method such as microbial conversion method, purification method from the microbial cultivates, enzymatic method or chemical synthetic method.

Preferable peptide polymers are any one which can be mixed with cosmetic, however, collagen, hydrolysable collagen, gelatin, elastin, hydrolysable gelatin, keratin etc. including the peptide polymer used in examples of present invention are preferable.

Preferable polysaccharide polymers are any one which can be mixed with cosmetic, however, hydroxy ethyl cellulose, xanthin gum, hyaluronic acid Na, chondroitin sulfate or their salt (Na salt etc) and the like are preferable. For example, chondroitin sulfate or the salt thereof etc can be used by being purified from mammal or fishes ordinarily.

Preferable sphingolipid are any one which can be mixed with cosmetic, however, ceramide, pit-sphingosin, sphingo-lipopolysaccharide and the like are preferable. Sphingo-lipid can be obtained by being purified from mammal, fish, shellfish, yeast or plant etc in conventional method.

Preferable seaweed extract is any one which can be mixed with cosmetic, however, the extract of brown algae, red algae, green algae and the like or the purified carrageenan, alginic acid, arginic acid Na, K isolated therefrom are preferable. Algae extract can be obtained by being purified from seaweed in conventional method.

The cosmetic composition of the present invention may combine with other ingredients used in conventional cosmetic composition, if necessary, together with above described essential ingredient.

Preferable above described other ingredients may comprise oil ingredient, humectants, emollients, surfactants, organic or inorganic dye, organic powder, ultraviolet ray absorbing agent, preservatives, antiseptics, antioxidants, plant extract, pH controller, alcohol, pigments, perfumes, refrigerants, blood circulator, antihidrotic, distilled water and etc.

Preferable oil ingredients may comprise ester oil, hydrocarbon oil, silicone oil, fluoride oil, animal oil, plant oil and so on.

Preferable ester oil described above may comprise glyceryl tri-2-ethyl hexanoic acid, cetyl 2-ethyl hexanoic acid, isopropyl myristic acid, butyl myristic acid, isopropyl palmitic acid, ethyl stearic acid, octyl palmitic acid, isocetyl isostearic acid, butyl stearic acid, ethyl linoleic acid, isopropyl linoleic acid, ethyl oleic acid, isocetyl myristic acid, isostearyl myristic acid, isostearyl palmitic acid, octyldodecyl myristic acid, isocetyl isostearic acid, diethyl sebacic acid, isopropyl adipic acid, isoalkyl neopetanoic acid, glyceryl tri(capryl, capric acid), trimethylopropane tri-2-ethyl hexanoic acid, trimethylopropane triisostearic acid, pentaerythritol tetra-2 ethyl hexanoic acid, cetyl caprylic acid, decyl lauric acid, hexyl lauric acid, decyl myristic acid, myristyl myristic acid, cetyl myristic acid, stearyl stearic acid, decyl oleic acid, cetyl licinoleic acid, isostearyl lauric acid, isotridecyl myristic acid, isocetyl palmitic acid, octyl stearic acid, isocetyl stearic acid, isodecyl oleic acid, octyldodecyl oleic acid, octyldodecyl linoleic acid, isopropyl isostearic acid, cetostearyl 2-ethyl hexanoic acid, stearyl 2-ethyl hexanoic acid, hexyl isostearic acid, ethylene glycol dioctanoic acid, ethylene glycol dioleic acid, propylene glycol dicapric acid, propylene glycol di(capryl, capric acid), propylene glycol dicaprylic acid, neopentylglycol dicapric acid, neopentylglycol dioctanoic acid, glyceryl tricaprylic acid, glyceryl triundecylic acid, glyceryl triisopalmitic acid, glyceryl triisostearic acid, octyldodecyl neopentanoic acid, isostearyl octanoic acid, octyl isononanoic acid, hexyldecyl neodecanoic acid, octyldodecyl neodecanoic acid, isocetyl isostearic acid, isostearyl isostearic acid, octyldecyl isostearic acid, polyglycerin oleanoic acid ester, polyglycerin isostearic acid ester, triisocetyl citric acid, triisoalkyl citric acid, triisooctyl citric acid, lauryl lactic acid, myristyl lactic acid, cetyl lactic acid, octyldecyl lactic acid, triethyl citric acid, acetyltriethyl citric acid, acetyl tributyl citric acid, trioctyl citric acid, diisostearyl maleic acid, di 2-ethylhexyl hydroxy stearic acid, 2-ethyl hexyl succinic acid, diisobutyl adipic acid, diisopropyl sebasinic acid, dioctylsebacinic acid, cholesteryl stearic acid, cholesteryl isostearic acid, cholesteryl hydroxy stearic acid, cholesteryl hydroxy stearic acid, cholesteryl oleic acid, dihydrocholesteryl oleic acid, pitsteryl isostearic acid, pitsteryl oleic acid, isocetyl 12-stealoyl hydroxy stearic acid, stearyl 12-stealoyl hydroxy stearic acid, isostearyl 12-stealoyl hydroxy stearic acid.

Preferable hydrocarbon oil described above may comprise squalene, liquid paraffin, α-olefin oligomer, isoparaffin, ceresin, paraffin, liquid isoparaffin, polybuden, microcrystalline wax, vaselin and the like.

Preferable silicone oil may comprisepolymethylsilicone, methylphenylsilicone, methylcyclopolysiloxane, octamethylpolysiloxane, decamethylpolysiloxane, dodecamethylcyclosiloxane, dimethyl siloxane-methyl cetyloxysiloxan copolymer, dimethyl siloxane-methyl stealoxysiloxane copolymer, alkyl modified silicone oil, amino modified silicone oil and the like.

Preferable fluoride oil can comprise perfluoropolyether and the like.

Preferable animal or plant oil can comprise avocado oil, almond oil, olive oil, sesame oil, rice husk oil, safflower oil, soy-bean oil, corn oil, rape oil, amygdalin oil, palm kernel oil, palm oil, pimaja oil, sunflower oil, fruit seed oil, cotton seed oil, coconut palm oil cucui nut oil, wheat embryo bud oil, rice embryo bud oil, sia butter, evening-primrose oil, marker daymia nut oil, medo home oil, egg yolk oil, lanolin, hempseed oil, mink oil, orange ruppy oil, hohoba oil, carnauba wax, liquid lanolin, solid pimaja wax and the like.

Preferable humectants can comprise water-soluble low molecular humectants, lipophilic low molecular humectants, water-soluble polymer and lipid soluble polymer.

Specifically, preferable water soluble low molecular humectants can comprise cerin, glutamine, sorbitol, mannitol, pyrrolidone-carboxylic acid Na, glycerin, propylene glycol, 1,3-butylene glycol, ethylene glycol, polyethylene glycol (polymerization index. >2), polypropyleneglycol (polymerization index >2), lactic acid, lactate salt and the like.

Preferable lipid soluble low molecular humectants can comprise cholesterol, cholesteryl ester and the like.

Preferable water soluble polymer can comprise carboxy vinyl polymer, poly asparaginic acid salt, tragacanth, xanthin gum, HMC (hydroxy methyl celluose), HEC (hydroxy ethyl celluose), HPC (hydroxy propyl celluose), carboxymethylcellulose, water soluble chitin, chitosan, dextrin and the like.

Preferable lipid soluble polymer can comprise polyvinylpyrrolidone-eicocene copolymer, polyvinylpyrrolidone-hexadecene copolymer, nitrocellulose, dextrin fatty acid ester, silicone polymer and the like.

Preferable emollients can comprise long chain acyl glutamic acid cholesteryl ester, cholesteryl hydroxy stearic acid, 12-hydroxy stearic acid, rogic acid, lanolin fatty acid cholesteryl ester and the like.

Preferable surfactant can comprise nonionic surfactants, anionic surfactants, cationic surfactants, ambivalent surfactants and the like.

Specifically, preferable non-ionic surfactants can comprise self-emulsified monostearic acid glycerin, propylene glycol fatty acid ester, glycerin fatty acid ester, polyglycerin fatty acid ester, sorbitan fatty acid ester, polyoxyethylene (POE) sorbitan fatty acid ester, POE sorbitan fatty acid ester, POE glycerin fattyacid ester, POE alkyl ether, POE fatty acid ester, POE solid pimaja oil, POE pimaja oil, POE-POP copolymer, POE-POP alkyl ether, polyether modified silicone, lauric acid alkanol amide, alkyl amine oxide, hydrogen addition soybean phospholipid and the like.

Preferable anionic surfactants can comprise fatty acid soap, -acyl sulfonic acid salt, alkyl sulfonic acid salt, alkyl ally sulfonic acid, alkyl naphthalene sulfonic acid salt, alkyl sulfonic acid salt, POE alkylether sulfate salt, alkyl amide sulfate salt, alkyl phosphate salt, POE alkyl phosphate salt, alkylamide phosphate salt, alkyloylalkyl taurine salt, N-acyl-amino acid salt, POE alkyl ether carboxylic acid salt, alkyl sulfo succinic aid salt, alkyl sulfo-acetic acid salt, acylated hydrolysable collagen peptide salt, perfluoro alkyl phosphate ester and the like.

Preferable cationic surfactant can comprise alkyl trimethyl ammonium chloride, stearyl trimethyl ammonium chloride, stearyl trimethyl ammonium bromide, setostearyltrimethyl ammonium chloride, distearyl dimethyl ammonium chloride, stearyl dimethyl benzyl ammonium chloride, vehenyltrimethyl ammonium bromide, benzalkonium chloride, diethylamino ethyl amide stearic acid, dimethylaminopropyl amide stearic acid, lanolin derivatives quaternary ammonium and the like.

Preferable ambivalent surfactants can comprise carboxy betaine type, amide betaine type, hydroxy sulfo betaine type, phosphobetaine type, aminocarboxylic acid, imidazoline derivatives type, amide amine type and the like.

Preferable organic and inorganic dyes can comprise silicic acid, anhydrous silicic acid, magnesium silicic acid, talc, ceracyte, mica, caolin, bengala, clay, bentonite, titan film mica, oxy chlorine bismuth, zirconium oxide, magnesium oxide, zinc oxide, titan oxide, aluminium oxide, calcium sulfate, barium sulfate, magnesium sulfate, calcium carbonate, magnesium carbonate, ferrous oxide, chromium oxide, chromium hydroxide, calamine, carbon black and the complex thereof as an inorganic dyes; polyamide, polyester, polypropylene, polystyrene, polyurethane, vinyl resin, urea resin, phenol resin, fluoride resin, silicone resin, acryl resin, melamine resin, epoxy resin, polycarbonated resin, divinyl benzene-styrene copolymer, silk powder, cellulose, CI pigment yellow, CI pigment orange as an organic dyes; and their complex etc.

Preferable organic powder can comprise metal soap such as calcium stearate; alkyl phosphonate metal salt such as sodium zinc cetylic acid, zinc laurylic acid, calcium laurylic acid; acylamino acid polyvalent metal salt such as calcium N-lauroyl-alanine, zinc N-lauroyl-alanine, calcium N-lauroyl-glycine etc.; amide sulfonic acid polyvalent metal salt such as calcium N-lauroyl-taurine, calcium N-palmitoyl-taurine; N-acyl basic amino acid such as Nc-lauroyl-L-lysine, Nc-palmitoyl-lysine, N-palmitoyl ornitine, N-lauroly arginine, hardened lanolin fatty acid acyl arginine and the like; N-acylpolypeptide such as N-lauroylglycyl glycine; -amino fatty acid such as amino caprylic acid, amino lauric acid and the like; polyethylene, polypropylene, nylon, polymethylmetacrylate, polystyrene, divinylbenzene-styrene copolymer, ethylene tetrafluoride and so on.

Preferable ultraviolet absorbing agents can comprise paraaminobenzoic acid, paraamonoethyl benzoate, paraamino amyl benzoate, paraamino octyl benzoate, ethyleneglycol salicylate, phenyl salicylate, octyl salicylate, benzyl salicylate, butylphenyl salicylate, homomentyl salicylate, benzyl cinnamic acid, paramethoxy 2-ethoxy ethyl cinnamic acid, paramethoxy octyl cinnamic acid, diparamethoxy mono-2-ethylhexane glyceryl cinnamic acid, paramethoxy isopropyl cinnamic acid, diisopropyl-diisopropyl cinnamate ester mixture, urokanic acid, ethyl urokanic acid, hydroxy methoxy benzophenone, hydroxymethoxy benzophenone sulfonic acid and their salt, dihydroxy methoxy benzophenone, dihydroxy methoxy benzophenone disulfonate Na, dihydroxy benzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl) benzotriazole and the like.

Preferable preservatives can comprise hinokitiol, trichloric acid, trichlorohydroxydiphenylether, chlorohexidine glucuronate, phenoxyethanol, resorcine, isopropylmethylphenol, azulene, salicylic acid, zinc pilithione, bezalconium HCl, photosensitizer 301, mononitroguaiacol Na, undecylenic acid etc.

Preferable antioxidants can comprise butylhydroxyanisole, propyl gallate, ellisorbate and the like.

Preferable pH controller can comprise citric acid, sodium citrate, malic acid, sodium malate, fumaric acid, sodium fumaric acid, succinic acid, sodium succinic acid, sodium hydroxide, sodium hydrogen phosphate and the like.

Preferable alcohol can comprise cetyl alcohol etc.

Furthermore, other ingredient addable to above described component and the amount thereof is not limited within the scope of the purpose and effect of the present invention, however, it is preferable that the amount of the other ingredients ranges from 0.01 to 10%, more preferably, 0.01 to 5% in that of total composition.

The cosmetic composition of the present invention can be modified as a solution, emulsion, cohesive mixture etc.

Above described ingredients such as water-soluble vitamin, lipid soluble vitamin, peptide polymer, polysaccharide polymer, sphingolipid, sea weed extract and addable ingredients which can be added other than above described ingredients if necessary, can be obtained by conventional methods disclosed in the literature (Matsumoto Mithio; Manual for the development of transdermal applied preparation. Seisi Press, 1^{st} Ed., 1985).

Specifically, cosmetic formulation of the present invention may be prepared in any form well-known in the art for example, skin lotion, skin softener, skin toner, astringent, lotion, milk lotion, moisture lotion, nutrient lotion, massage lotion, nutrient cream, moisture cream, hand cream, foundation, essence, nutrient essence, pack, soap, cleansing foam, cleansing lotion, cleansing cream, body solution, body cleanser and the like.

More specifically, the paste, cream or gel formulation of the present invention may use lactose, talc, silica, aluminum hydroxide, calcium silicate or polyamide powder etc. as a cosmetic carrier and further add chlorofluorohydrocarbon, propane/butane or dimethyl ether as an expellant.

More specifically, the solution of emulsion formulation of the present invention may use solvent, solubilizer, or emulsifier such as water, ethanol, isopropanol, ethyl carbonate, ethyl acetate, benzyl alcohol, benzyl benzoate, propylene glycol, 1,3-butylene glycol, glycerol fatty ester, PEG or sorbitan fatty ester etc.

More specifically, the suspension formulation of the present invention may use appropriate carrier, for example, liquid dilution such as water, ethanol or PEG emulsifier such as ethoxylated isostearyl alcohol, polyoxyethylene sorbitol ester, or polyoxyethylene sorbitane ester; and microcrystalline cellulose, aluminum meta hydroxide, bentonite, agar or traganth etc.

More specifically, the surfactant comprising cleansing formulation of the present invention may use fatty alcohol sulfate, fatty alcohol ether sulfate, sulfosuccinic acid monoester, icetionate, imidazolinum derivative, methyl taurate, sarocynate, fatty acid amide ethyl sulfate, alkyl amido betaine, fatty alcohol, fatty acid glyceride, fatty acid diethanol amide, plant oil, linolic acid, or ethoxylated glycerol fatty acid ester etc.

Inventive compounds of the present invention have no toxicity and adverse effect therefore can be used with safe.

### Advantageous Effects

The present invention relates to a composition comprising an extract of combined herbs consisting of heat processed ginseng, Houttuyniae Herba, Perilla leaf and Tea leaf or the processed extract thereof showing preventing activity of baldness disorder and stimulating activity of hair growth.

The inventive combined extract showed more potent hair-growth promoting activity and synergistic effect than the precedent invention(s), for example, a fermented extract of Houttuyniae Herba, Perilla leaf and Tea leaf disclosed in Korea Patent Publication No, 10-2014-0114492 (A1), through various animal model experiments such as the growth rate test using by C57BL/6 mouse, the proliferating effect on the growth of HFDPC etc and additionally, found more favorable advantage than the precedent invention(s), for example, the easiness in controlling the prescribed dosage by dint of the final form of the inventive extract, i.e., concentrated solid form comparing with the solution type of the final form disclosed in precedent invention.

### DESCRIPTION OF DRAWINGS

### Best Mode

The above and other objects, features and other advantages of the present invention will more clearly understood from the following detailed description taken in conjunction with the accompanying drawings, in which;

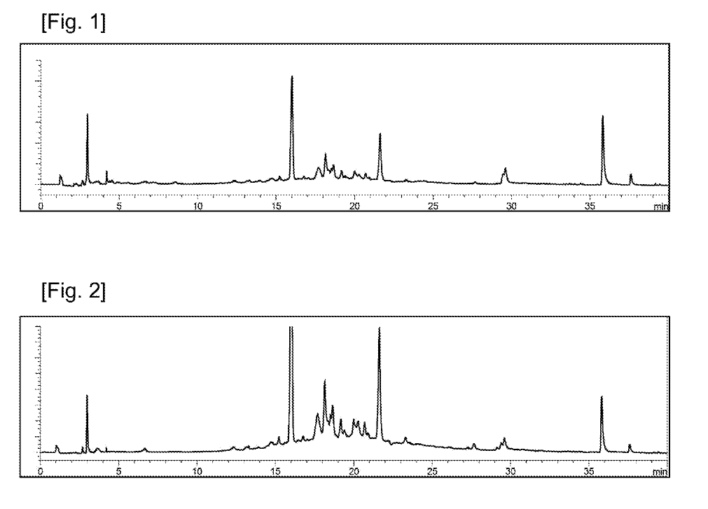

FIG. 1 shows the analysis result of HPLC analysis of the inventive combined extract;

FIG. 2 shows the analysis result of HPLC analysis of the inventive combined processed extract.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Comparative Example 1

### Preparation of the extract disclosed in precedent invention (KR 10-2014-0114492 A1)

In accordance with the method disclosed in KR 10-2014-0114492 (A1), 100 g of dried houttuyniae herba, 60 g of dried perilla leaf and 40 g of tea leaf were pulverized and 4.0 g of yeast was added thereto to mix together and be sealed hermetically. After 2 days the sealing, 3.2 L of 30% spirit was poured into the mixture to stir gently and the bottle containing the solution was sealed hermetically. The bottle was kept in the dark room maintaining the temperature of about 20° C. and relative humidity of about 55% for 6 months to ferment the solution. During the fermentation, the solution was stirred to mix thoroughly for 10 mins, once a week. 6 weeks later, the bottle was open and the fermented solution was filtered to remove debris and to collect 3.0 L of fermented extract. The fermented extract was concentrated to obtain 40 g of final fermented extract of combined herbs consisting of houttuyniae herba, perilla leaf and tea leaf (designated as “RCF” hereinafter), which was used as a comparative sample in the following Experimental Examples.

### Example 1

### The Preparation of Inventive Extract

### 1-1. Preparation of Extract of Heat Processed Ginseng (SG)

The heat processed ginseng was prepared according to the method disclosed in Korean Patent Registration No. 192678 and U.S. Pat. No. 5,776,460 as follows:

100 g of dried ginseng radix was heated at 120° C. for 3 hours and dried at 60° C. for one night to obtain 33 g of a heat processed ginseng radix (designated as “SG” hereinafter). 10 g of the heat processed ginseng was mixed with 8 fold weight of 70% (v/v) ethanol and the solution was extracted for 2 hours with reflux extraction at above 90° C. The resulting residue was filtered with a filter paper (Whatman Co.) and the filtrate was concentrated with rotary evaporator (Heidolph, Laboota 4001). The concentrated extract was dried with freeze dryer (FDU-210, EYELA) to obtain 4.0 g of powdered extract of heat processed ginseng radix (designated as “SGE” hereinafter).

### 1-2. Preparation of Inventive Extract of Combined Herbs (CHE)

20 g of the SG heat processed ginseng radix prepared in Step 1-1 as well as 100 g of Houttuyniae Herba, 60 g of Perilla leaf and 40 g of tea leaf were pulverized and mixed together. 4 L of 70% (v/v) ethanol was added thereto and the solution was extracted for 1 hour with reflux extraction at above 90° C. The resulting residue was filtered with a filter paper (Whatman Co.) and the filtrate was concentrated with rotary evaporator (Heidolph, Laboota 4001). The concentrated extract was dried with freeze dryer (FDU-210, EYELA) to obtain 84.5 g of inventive extract of four herbs (designated as “CEH” hereinafter), which was used as a test sample in the following Experimental Examples.

### Example 2

### The Preparation of Inventive Combined Processed Extract

5 g of the CHE extract prepared in Example 1 was performed to ion exchange column chromatography by using DIAION HP-20 column (Samyang. Co. Ltd., Korea) as a stationary phase with eluting with water in order to adsorbing non-polar substances onto the resin and ethanol to collect the adsorbed non-polar substance. The collected elute was concentrated with rotary evaporator (Heidolph, Laboota 4001) and dried with freeze dryer (FDU-210, EYELA) to obtain 2.2 g of inventive resin-treated extract of four herbs (designated as “CEHR” hereinafter), which was used as a test sample in the following Experimental Examples.

### Experimental Example 1

### HPLC component analysis

To analyze the change of component in CEH extract and CEHR extract prepared in Examples, the following HPLC (high performance liquid chromatography) analysis was performed according to the condition disclosed in following Table 1.

TABLE 1
Condition of HPLC analysis

| Pump | Agilent 1260 series, 1260 quart pump | | |
|---|---|---|---|
| Detector | Agilent 1260 series, 1260 DAD | | |
| Column | Agilent Eclipse XDB C18, 4.6 × 250 mm, 5 μm | | |
| Flow rate | 1.0 ml/min | | |
| UV absorbance | 266 nm | | |
| | Mobile phase A: acetate buffer solution (pH = 3.5) | | |
| | Mobile phase B: acetonitrile | | |
| | | Mobile phase A | Mobile phase B |
| Mobile phase | Time | (%) | (%) |
| | 0-5 | 80 | 20 |
| | 5-20 | 75 | 25 |
| | 20-25 | 75 | 25 |
| | 25-30 | 55 | 45 |
| | 30-35 | 55 | 45 |
| | 35-36 | 80 | 20 |
| | 36-40 | 80 | 20 |

| | |
|---|---|
| Injection volume | 10 μL |

As can be seen in FIGS. 1 and 2, it has been confirmed that the CEHR extract treated with HP resin contains more abundant active ingredients which had been adsorbed onto HP-20 resin, i.e., non-polar substances, compared with CEH extract containing ineffective ingredients such as polysaccharides.

### Experimental Example 2

### Promoting Effect on Hair Growth

To confirm the effect of inventive extract on the preventing activity of baldness disorder and stimulating activity of hair growth, the following method was performed according to the method disclosed in the literature (Rho S S et al., J. Dermatol. Sci., 38(2), pp 89-97, 2005).

### 2-1. Preparation

6 weeks-old mouse (C57BL/6, 18-20 g, at the beginning of catagen, Orient Bio Inc. Seoul, Korea) was accustomed to the breeding environment for 1 week and anesthetized intramuscularly with an anesthetizer (2.5 mg/mouse, Ketamine, rompun, Bayer Korea Inc.). The back-hair of anesthetized mouse was removed by a grainer and the mice were divided into four groups consisting of 10 mice, respectively, i.e., (1) negative control group: treatment group with only solvent, (2) positive control group: treatment group with Minoxidil (0.5%, w/w), (3) comparative test group: treatment group with the extract prepared in Comparative Example 1 dissolved in adjuvant in a dose dependent manner, (4) test sample group: treatment group with the extract prepared in Examples dissolved in adjuvant in a dose dependent manner. 2.5 mg of the test samples was spread on the back of mouse once a day for 20 days.

### 2-2. Hair-Growth Promoting Effect

The ratio of hair-regrown area/shaved area was quantitatively analyzed by image analyzer (Image-Pro, USA) after shooting the hair-grown area with digital camera (Canon, Japan) and the test result was scored to 5 scores, i.e., score 0: ineffective; score 1: merely effective; score 2: favorably effective; score 3; potently effective; score 4; most potently effective as shown in Table 2.

TABLE 2
scored criteria for determination

| Hair growth ratio | score | Criteria (Abbr.) |
|---|---|---|
| 0-20% | 0 | Ineffective (IE) |
| 20-40% | 1 | merely effective (ME) |
| 40-60% | 2 | favorably effective (FE) |
| 60-70% | 3 | potently effective (PE) |
| >70% | 4 | most potently effective |
| | | (MPE) |

At the result, it has been confirmed that the inventive extracts, i.e., CEH and CEHR extract, have more potent treating effect on alopecia compared with the comparative test group treated with RCF extract disclosed in KR 10-2014-0114492 (A1), as well as negative control group treated with only solvent and positive control group treated with Minoxidil (0.5%, w/w) from 15 days after the beginning of experiment, as can be seen in following Table 3.

TABLE 3
Hair-growth activity

| | | Negative | | | | | Positive |
|---|---|---|---|---|---|---|---|
| Day | Item | control | SGE | CEH | CHER | RCF | control |
| 5^{th} day | Hair | 9% | 12% | 12% | 12% | 10% | 14% |
| | growth | | | | | | |
| | ratio | | | | | | |
| | Score | 0 | 0 | 0 | 0 | 0 | 0 |
| | Criteria* | IE | IE | IE | IE | IE | IE |
| 10^{th} day | Hair | 22% | 28% | 42% | 50% | 38% | 42% |
| | growth | | | | | | |
| | ratio | | | | | | |
| | Score | 1 | 2 | 2 | 2 | 1 | 2 |
| | Criteria | ME | ME | FE | FE | ME | FE |
| 15^{th} day | Hair | 28% | 39% | 68% | 74% | 55% | 62% |
| | growth | | | | | | |
| | ratio | | | | | | |
| | Score | 1 | 3 | 3 | 4 | 2 | 3 |
| | Criteria | ME | ME | PE | MPE | FE | PE |
| 20^{th} day | Hair | 38% | 48% | 76% | 84% | 68% | 68% |
| | growth | | | | | | |
| | ratio | | | | | | |
| | Score | 1 | 4 | 4 | 4 | 3 | 3 |
| | Criteria | ME | ME | MPE | MPE | PE | PE |

*criteria (Abbr.): IE (Ineffective), ME (Merely effective), FE (favorably effective), PE (potently effective), MPE (most potently effective)

### Experimental Example 3

### Proliferating Effect on HFDPC

To confirm the effect of inventive extract on the proliferating effect on HFDPC (Hair Follicle Dermal Papilla Cell) cell, the following method was performed according to the already known method disclosed in the literature (Korea Patent Registration No. 10-1443142 B1).

### 3-1. 1^{St} Step; HFDPC Cell Culture

HFDPC (Hair Follicle Dermal Papilla Cell) cells (ATCC) were seeded onto T75 plate (FA3024, Falcon) at a density of 2×10⁵ cells/plate and cultivated in S(+) supplement mix culture media containing IX antibiotic-anti-mycotic (LS203-01, Welgene Inc.) for 4-5 days.

After the culture, the cells were transferred to 48 well plates (30048, SPL Life Sciences) at a density of 6000 cells/well and cultured in S(−) supplement free culture medium (Welgene Inc.) for 1 day.

### 3-2. 2^{nd} Step; Sample Treatment

After the cells had adhered to the wells, the S(+) growth media was removed and various concentration of the test samples (CEH and CEHR) were diluted with S(−) supplement free culture medium to divide to five groups, i.e., (1) negative control group treated with only S(−) media, (2) test sample groups treated with 40 μg/ml of test sample, (3) test sample groups treated with 80 μg/ml of test sample, (4) test sample groups treated with 160 μg/ml of test sample, and (5) positive control group treated with only S(+) supplement mix culture media.

The media in each well was changed with new media at the interval of 24 hrs and 48 hrs and 72 hrs after the treatment, the cell proliferation of each group was determined.

The cell proliferation of comparative test group treated with RCF extract was also determined according to the similar procedure to the above.

3-3. 3^{rd} step; determination of HFDPC proliferation

The proliferation of HFDPC cell was determined using by EZ-cytox kit (EZ-3000, DAEILLAB Service Co., Ltd., Korea), a cell viability response kit using by WST (high sensitive water soluble terazolium salt) which has widely used to specifically determine the condition of cells by way of reacting with all the dehydrogenase enzymes in the cell (EZ-Cytox Manual, DAEILLAB Service Co., Ltd., Korea).

After displacing culture media in each well with new S(−) media with a volume of 200 μL/well in 48 well plates. 20 μL/well of the reaction solution provided from EZ-cytox kit (EZ-3000, DAEILLAB Service Co., Ltd., Korea) was treated to each cell and the cell was cultivated for 2 hours in 5% CO2 incubator (311, Forma Co.) at 37° C. The absorbance at 450 nm of each group was determined by microplate reader (SpectraMax 340 PC, Molecular Device) and the number of each cell was calculated and analyzed based on the value of O.D. ratio.

The activating effect of each group on the proliferation of HFDPC cell was shown in Table 4.

TABLE 4
HFDFC cell proliferation activity

| treatment | SGE | CEH | CHER | RCF |
|---|---|---|---|---|
| S(−)* | 100.0% | 100.0% | 100.0% | 100.0% |
| S(−) + 40 | 105.2% | 119.2% | 132.1% | 110.7% |
| μg/ml** | | | | |
| S(−) + 80 | 107.3% | 128.6% | 148.9% | 114.6% |
| μg/ml** | | | | |
| S(−) + 160 | 113.5% | 132.9% | 160.4% | 120.8% |
| μg/ml** | | | | |

*S(−): negative control group treated with S(−) media
**S(−) + 40-160 μg/ml: various concentrations of test sample groups with S(−) media

At the result, it has been confirmed that the test group treated with 40 μg/ml, 80 μg/ml, and 160 μg/ml of CEH extract or CHER extract showed more increased proliferation of HFDFC cell by 19.2%, 28.6%, and 32.9% (CEH extract) or by 32.1%, 48.9%, and 60.4% (CHER extract), compared with negative control group and the test group treated with inventive extract showed unexpectedly more potent increasing effect on proliferation of HFDFC cell, compared with comparative example group treated with RCF extract, which means that the inventive extract more potently activated the proliferation of HFDFC cell and showed potent treating effect on alopecia, as can be seen in Table 4.

Accordingly, it has been confirmed that the inventive extract has potent promoting effect on hair-growth and has various advantages such as safe and little adverse response compared with the conventionally, available chemical drugs with various disadvantage.

Hereinafter, the formulating methods and kinds of excipients comprising the inventive extract of the present invention, will be described, but the present invention is not limited to them. The representative preparation examples are described as follows.

### Preparation of Powder

| | | |
|---|---|---|
| Extract (CEH) | 500 | mg |
| Lactose | 100 | mg |
| Talc | 10 | mg |

Powder preparation was prepared by mixing above components and filling sealed package.

### Preparation of Tablet

| | | |
|---|---|---|
| Extract (CHER) | 500 | mg |
| Corn Starch | 100 | mg |
| Lactose | 100 | mg |
| Magnesium Stearate | 2 | mg |

Tablet preparation was prepared by mixing above components and entabletting.

### Preparation of Capsule

| | | |
|---|---|---|
| Extract (CEH) | 500 | mg |
| Crystallized cellulose | 3 | mg |
| Lactose | 14.8 | mg |
| Magnesium Stearate | 0.2 | mg |

Capsule preparation was prepared by mixing above components and filling gelatin capsule by conventional gelatin preparation method.

### Preparation of Injection

| | | |
|---|---|---|
| Extract (CHER) | 100 | mg |
| Mannitol | 180 | mg |
| Distilled water for | 2974 | mg |
| injection | | |
| Na₂HPO₄•12H₂O | 26 | mg |

Injection preparation was prepared by dissolving active component and then filling all the components in 2 ml ample and sterilizing by conventional injection preparation method.

### Preparation of Liquid

| | | |
|---|---|---|
| Extract (CEH) | 1000 | mg |
| Isomerized sugar | 10 | g |
| Mannitol | 5 | g |

| | |
|---|---|
| Distilled water | optimum amount |

Liquid medicine was prepared by dissolving the components to distilled water with a proper dose of lemon scent, mixing, adjusting to 100 ml with distilled water in brown bottle and sterilizing by conventional liquid medicine preparation method.

### Preparation of Health Functional Food

| Extract (CHER) | 500 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 100 | mg |
| Magnesium chloride | 24.8 | mg |

The above-mentioned vitamin and mineral mixture may be varied in many ways. Such variations are not to be regarded as a departure from the spirit and scope of the present invention.

### Preparation of Health Beverage

| | | |
|---|---|---|
| Extract (CEH) | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirring at 85° C. for 1 hour, filtering and then filling all the components in 2 t container and sterilizing by conventional health beverage preparation method.

| Extract (CHER) | 0.5% (w/w) |
|---|---|
| white vaseline | 2.5% (w/w) |
| stearyl alcohol | 0.22% (w/w) |
| ethyl (or methyl) | 0.25% (w/w) |
| p-oxybenzoate | |
| propylene glycol | 12.0% (w/w) |
| sodium lauryl sulfate | 0.15% (w/w) |
| propyl p-oxybenzoate | 0.15% (w/w) |
| Distilled water up to | 100% |

Ointment preparation was prepared by mixing the above components, filling in a ointment tube by conventional ointment preparation method.

### Preparation of Hair Tonic

| | |
|---|---|
| Extract (CEH) | 10.00% |
| menthol | 0.05% |
| Panthenol | 0.2% |
| salicylic acid | 0.1% |
| tocopherol acetate | 0.1% |
| salicylic acid | 0.1% |
| pyridoxine•HCl | 0.1% |
| castor oil | 5.0% |
| pigment optimum amount | |
| Flavour-optimum amount | |
| ethanol-optimum amount | |
| Distilled water up to | 100% |

Hair tonic preparation was prepared by dissolving the active components according to conventional hair tonic preparation method.

### Preparation of Skin Conditioner

| | |
|---|---|
| Extract (CHER) | 2.5% |
| cetanol | 3.5% |
| self-emulsifying | 1.5% |
| mono-stearate glycerol | |
| Propylene glycol | 2.5% |
| stearyl methyl benzyl | 7.0% |
| ammonium chloride (25%) | |
| methyl p-oxybenzoate | 0.3% |
| | (w/w) |
| pigment-optimum amount | |
| Flavour-optimum amount | |
| Distilled water up to | 100% |

Skin conditioner preparation was prepared by dissolving the active components according to conventional skin conditioner preparation method.

### Preparation of Hair Lotion

| | |
|---|---|
| Extract (CEH) | 5.00% |
| resorcinol | 2.00% |
| menthol | 2.00% |
| Panthenol | 0.5% |
| piroctone olamine | 0.1% |
| Flavour & pigment | 0.5% |
| Distilled water up to | 100% |

Hair lotion preparation was prepared by dissolving the active components according to conventional hair lotion preparation method.

### Preparation of Hair Soap

| | |
|---|---|
| Extract (CHER) | 0.1% |
| titanium dioxide | 0.2% |
| polyethylene glycol | 0.8% |
| glycerin | 0.5% |
| ethylene diamine tetracetate | 0.05% |
| sodium | 1.00% |
| pigment-optimum amount | |
| soap flavour-optimum amount | |
| beauty soap base | |
| (humidity 13%) up to | 100% |

Hair soap was prepared by dissolving the active components according to conventional hair soap preparation method.

### Preparation of Cream

| | |
|---|---|
| Extract (CEH) | 3.00% |
| Polyethyleneglycomonosterate | 2.00% |
| Monostearate glycerin | 1.00% |
| Cetyl alcohol | 4.00% |
| Squalene | 6.00% |
| Tri 2-glycerly ethylhexanoate | 6.00% |
| Sphingo-glycolipid | 1.00% |
| 1,3-butylene glycol | 3.00% |
| Distilled water up to | 100% |

Cream preparation was prepared by dissolving the active components according to conventional cream preparation method.

### Preparation of Beauty Solution

| | |
|---|---|
| Extract (CHER) | 2.00% |
| Hydroxyethylene cellulose | 12.00% |
| (2% solution) | |
| Xanthin gum | 2.00% |
| (2% solution) | |
| 1,3-butylene glycol | 3.00% |
| Glycerin concentration | 4.00% |
| Sodium hyaluronte | 5.00% |
| Distilled water | 100 ml |

Beauty solution preparation was prepared by dissolving the active components according to conventional beauty solution preparation method

The invention being thus described as will be obvious that it may be varied in many ways. Such variations are not to be regarded as a departure from the spirit and scope of the present invention, and all such modifications as would be obvious to those skilled in art are intended to be included within the scope of the following claims.

### INDUSTRIAL APPLICABILITY

As described in the present invention, the present invention relates to a composition comprising the extract of combined herbs consisting of heat processed ginseng, houttuyniae herba, perilla leaf and tea leaf or the processed extract thereof as active ingredients for preventing and treating hair baldness and stimulating activity of hair growth.

The inventive combined extract showed more potent hair-growth promoting activity and synergistic effect than the precedent invention(s), for example, a fermented extract of Houttuyniae Herba, Perilla leaf and Tea leaf disclosed in Korea Patent Publication No, 10-2014-0114492 (A1), through various animal model experiments such as the growth rate test using by C57BL/6 mouse, the proliferating effect on the growth of HFDPC etc and additionally, more favorable advantage than the precedent invention(s), for example, the easiness in controlling the prescribed dosage by dint of the final form of the inventive extract, i.e., concentrated solid form comparing with the solution type of the final form disclosed in precedent invention.

1. A pharmaceutical composition for treating hair baldness and stimulating activity of hair growth comprising an extract of heat processed ginseng, houttuyniae herba, perilla leaf and tea leaf, or a processed extract thereof.

2. The pharmaceutical composition according to claim 1, wherein said heat processed ginseng is selected from the group consisting of root, leaf, fruit, and rhizome of a red ginseng or the processed ginseng produced by heating at a temperature ranging from 80 to 200° C. for a period ranging from 0.5 to 78 hours, so as to make a ratio of ginsenoside (Rg3+Rg5+Rk1) to (Rb1+Rb2+Rc+Rd) of over 1.0, wherein the red ginseng is selected from the group consisting of Panax ginseng, Panax quinquefolius, Panax notoginseng, Panax japonica, Panax trifolia, Panax pseudoginseng, Panax vietnamensis, Panax elegatior, Panax wangianus and Panax bipinratifidus.

3. The pharmaceutical composition according to claim 1, wherein said extract is an extract soluble in at least one solvent selected from the group consisting of water, ethanol, butanol, propanol, acetone, ethylacetate, hexane, butyleneglycol, propyleneglycol, hydrobutyleneglycol, hydropropyleneglycol, hydroglycerin, and mixtures thereof.

4. The pharmaceutical composition according to claim 1, wherein said extract of heat processed ginseng, houttuyniae herba, perilla leaf and tea leaf has a ratio of 1-10:1-10:110:1-10 (w/w).

5. The pharmaceutical composition according to claim 1, wherein said processed extract is a processed extract with reverse phase partition chromatography or any chromatography using any resin as a stationary phase; which in combination with a solvent can retain a non-polar substance while eluting a polar substance.

6. The pharmaceutical composition according to claim 1, wherein said baldness is selected from the group consisting of androgenic alopecia, alopecia senile, and alopecia areata.

7. A method for preparing the extract or the processed extract thereof according to claim 1 comprising the steps of;
heating a dried ginseng with high temperature ranging from 80 to 200° C. for a period ranging from 0.5 to 78 hours, so as to make a ratio of ginsenoside (Rg3+Rg5+Rk1) to (Rb1+Rb2+Rc+Rd) of over 1.0 to afford a heat processed ginseng at the 1^{st} step;
mixing the heat processed ginseng with houttuyniae herba, perilla leaf and tea leaf with a mixed ratio of 1-10:1-10:1-10:1-10 (w/w), to afford combined herbs at the 2^{nd} step;
mixing the combined herbs with at least one solvent selected from the group consisting of water, ethanol, butanol, propanol, acetone, ethylacetate, hexane, butyleneglycol, propyleneglycol, hydrobutyleneglycol, hydropropyleneglycol, hydroglycerin, and mixtures thereof, and subjecting to an extraction selected from the group consisting reflux extraction, cold water extraction, ultra-sonication and other conventional extraction methods with a temperature ranging from 0 to 150° C. for a period ranging from 0.1 to 48 hours, 1-10 times to afford the extract of combined herbs at the 3^{rd} step;
filtering and concentrating the extract at the 4^{th} step;
suspending the concentrating extract into water to afford the suspend solution at the 5^{th} step;
performing the suspended solution with a reverse phase partition chromatography using at least one resin selected from the group consisting of Sephadex, Sephadex LH20, Sephadex G-25, Sephadex G-10, Sepharose, Superdex, methylacrylate resin, carboxymethyl cellulose, sulphopropyl cellulose, carboxymethyl Sephadex, sulphopropyl Sephadex, carboxymethyl Sepharose, and sulphopropyl Sepharose as a stationary phase and using at least one solvent selected from the group consisting of water, acetonitrile, methanol, ethanol, butanol, tetrahydrofuran (THF) and mixtures thereof as a mobile phase to elute a polar substance and to collect a non-polar soluble fraction from the extract at the 6^{th} step; and
concentrating the non-polar soluble fraction to afford the processed extract of combined herbs according to claim 1.

8-11. (canceled)

12. A method of treating a baldness disorder in a mammal or human in need thereof comprising administering to said mammal or human an effective amount of an extract of heat processed ginseng, houttuyniae herba, perilla leaf and tea leaf or a processed extract thereof, together with a pharmaceutically acceptable carrier thereof.

13. The pharmaceutical composition according to claim 5, wherein the resin is selected from the group consisting of Sephadex, Sephadex LH20, Sephadex G-25, Sephadex G-10, Sepharose, Superdex, methylacrylate resin, carboxymethyl cellulose, sulphopropyl cellulose, carboxymethyl Sephadex, sulphopropyl Sephadex, carboxymethyl Sepharose, and sulphopropyl Sepharose.

14. The pharmaceutical composition according to claim 5, wherein the resin is a Stylene-divinylbenzen co-polymer reverse polymer resin selected from the group consisting of Polymer X, HP20, and PRP-h1 Polymer.

15. The pharmaceutical composition according to claim 5, wherein the resin is a Methacrylate support resin.

16. The pharmaceutical composition according to claim 5, wherein the solvent is at least one solvent selected from the group consisting of water, acetonitrile, lower alcohol, methanol, ethanol, butanol, tetrahydrofuran (THF) and mixtures thereof, to elute the polar substance and retain the non-polar substance.

17. The pharmaceutical composition according to claim 16, wherein the solvent is at least one solvent selected from the group consisting of water, lower alcohol, methanol, ethanol, butanol and mixtures thereof.

18. The pharmaceutical composition according to claim 17, wherein the solvent is elected from the group consisting of water and a mixture of water and ethanol.

19. The pharmaceutical composition according to claim 18, wherein the mixture of water and ethanol has a ratio ranging from 90:10 (v/v) to 60:40(v/v).
